(19) United States
(12) Patent Application Publication
(10) Pub. No.: US 2019/0298894 A1
(43) Pub. Date: Oct. 3, 2019

(54) SUCTION TIP FOR GENTLY SUCTIONING A BIOLOGICAL FLUID

(71) Applicant: Georg-August-Universitaet Goettingen Stiftung Oeffentlichen Rechts, Universitaetsmedizin, Goettingen (DE)

(72) Inventor: Martin Friedrich, Bovenden (DE)

(21) Appl. No.: 16/445,799

(22) Filed: Jun. 19, 2019

Related U.S. Application Data
(63) Continuation of application No. PCT/EP2017/084321, filed on Dec. 22, 2017.

(30) Foreign Application Priority Data
Dec. 23, 2016 (DE) 10 2016 125 556.4

(51) Int. Cl. A61M 1/00

(52) U.S. Cl. CPC A61M 1/008; A61M 1/0031; A61M 2206/11

(57) ABSTRACT

The invention relates to a suction tip (1) for suctioning a biological fluid, comprising a main body (12) having a main shaft (3) and a wall (4), a connection (5) for a suction line (2) formed on the main shaft (3) on the main body (12) and defining a free connection cross-section (6) of the suction tip (1), and multiple decentralized suction holes (7) laterally entering the main body (12), running through the wall (4) of the main body (12) and communicating with the connection (5) in the main body (12), wherein, with the continuous extension of its path (13) through the wall (4) of the main body (12), a free minimum cross-section (10) of each decentralized suction hole (7) overlaps completely with the free connection cross-section (6) of the connection (5).

### CROSS REFERENCE TO RELATED APPLICATIONS

This application is a continuation to international application PCT/EP2017/084321 with an international filing date of Dec. 22, 2017 entitled “Suction tip for the gentle suctioning of a biological fluid” and claiming priority to co-pending German patent application DE 10 2016 125 556.4 entitled “Saugerspitze zum schonenden Absaugen von Blut aus Operationsfeldern” and filed on Dec. 23, 2016.

### FIELD OF THE INVENTION

The present invention relates to a suction tip for suctioning a biological fluid. Particularly, the invention relates to a suction tip comprising a main body having a main axis and a wall, a connector for a suction line being formed at the main body on the main axis and defining a free connection cross section, and a plurality of decentral suction holes laterally entering the main body, extending through the wall of the main body and communicating with the connector within the main body.

A biological fluid is to be understood as a fluid which is of biological origin or which contains biological material like biological cells, particularly living biological cells or larger organic molecules, like for example antibodies, oligopeptides, proteins or DNA, which are relevant for biological processes. The biological fluid to be sucked off may specifically be a body fluid of an animal or a human. The body fluid may be sucked off in the course of a medical intervention. Even more specifically, the biological fluid to be sucked may be blood. The blood may particularly be sucked off in an operating field.

So far as an animal is mentioned here and in other parts of this application this shall imply that the animal is no human.

When sucking blood off in operating fields to return the blood to the patient at which the respective operation is made, various problems occur, i. a. mixing the blood with bubbles of ambient, damages to components of the blood, particularly to red blood cells, white blood cells, platelets and thrombocytes, by shearing forces and a suction tip adhering at adjacent tissue of the patient. Ambient air mixed with the blood has to be removed before the blood may be returned to the patient to avoid air embolisms. If blood damaged by shearing forces is returned to a patient, this may result in incalculable damages, i.e. renal failure, lung damages, thrombosis, wound healing disorders and systemic inflammation reactions. A suction tip adhering to tissue is associated with the danger of damaging the tissue. Further, with an intermittingly adhering suction tip very high shearing forces may be applied to the blood being sucked off, whereas a permanently adhering suction tip is out of function.

Similar problems occur with sucking-off other biological fluids, like inter alia undesired activations, erroneous activations, structure changes to molecules like folding, denaturation, disintegration and the like.

### BACKGROUND

A suction device for suctioning liquids, particularly for sucking blood off operating fields is known from WO 2012/092948 A1 and US 2013/0324954 A1 belonging to the same patent family. This known suction device comprises a suction element which is provided with at least one suction opening for receiving the liquid, and a pump which is connected to the suction element and which is equipped to generate a vacuum in the suction element. The suction power acting at the suction opening is controlled by a control device dependent on signals from an acoustic wave sensor detecting acoustic waves which are generated by the suction element. If the control device by means of the acoustic wave sensor detects a characteristic acoustic wave pattern of a slurping sound, it reduces the suction power acting at the suction opening, because the slurping sound indicates unfavorable suction conditions which are associated with the danger of inter alia damages to blood components, and damages to blood cells or other biological substances. Additionally, the control device is connected to an adherence-suction sensor, and upon acknowledging an adherence-suction of the suction element by means of the adherence-suction sensor it reduces the suction power acting at the suction opening.

US 2014/0276486 A1 discloses a cardiotomy suction tube system with multiple tips. One of these suction tips has a hollow body with a plurality of apertures which allow fluid communication between the surrounding environment and an interior of the hollow body. The suction tip also has a cylindrical projection which extends outwardly from the hollow body. The cylindrical projection may receive or may be received by a suction line. The hollow body is generally capsule-shaped, and it is elongated with rounded ends. The cylindrical projection extends longitudinally of the hollow body from one of the rounded ends thereof. One aperture of the main body is provided at a distal end of the main body, the other apertures are provided at a circumference of the main body.

U.S. Pat. No. 7,955,318 B1 discloses a multi-purpose large bore medical suction system with suction devices that may be used to remove material from body cavities during medical procedures. The system is designed to adapt to large bore medical vacuum sources such as the large bore port of a collection canister. The system comprises a series of interchangeable tips including a means for endotracheal-catheter suctioning. A blunt tip suction component of the large bore medical suction system comprises a plurality of narrow-bore inlets, including distal tip inlets and side inlets forming a fine screen-like suction field or suction screen. The inlet pattern of the distal tip and sides of the blunt tip suction component are adapted to provide an atraumatic-suctioning of wide surface areas.

WO 88/00481 A1 discloses a surgical suction device with a perforated suction tip for removal of surgical debris with reduced clogging and with minimum trauma to tissue. Suction forces are arranged on the tip so that suction ports which remain unblocked when surgical debris lodges in other suction ports operate as a vacuum modulator facilitating the removal of the blockage. Further, the likelihood of blocking every suction port thereby aspirating and damaging tissue, is greatly reduced. Particularly, suction ports are both provided at the front and at the back of the bulbous suction tip, which lead to a center of the bulbous suction tip.

U.S. Pat. No. 5,827,218 A discloses a suction tip for a surgical irrigation apparatus. The suction tip includes an outer tube having a distal end portion for communication with a surgical site and a proximal end for communication with a suction irrigation hand piece, and an inner tube extending in the outer tube and having an open distal end portion for communication with a surgical site and an open proximal end portion for communication with the hand piece. A one way seal is interposed between the inner and outer tubes of the tip. A turbulence minimizing distal end portion of the outer tube comprises a convexly rounded end having a forward facing central opening and circumferentially and axially space suction flow holes opening through the wall of the outer tube into the hollow interior thereof. The holes are provided in eight axially extending, circumferentially spaced rows, each row including one forward facing hole in the rounded end and six holes facing radially outward in the tubular sleeve in the forward portion thereof immediately behind the rounded end. The suction flow of fluent material rearwardly into the multiplicity of holes minimizes turbulence and risk of damage of sensitive organs during suction.

There still is a need of a suction tip for suctioning a biological fluid whose geometric design minimizes the problems which have been explained at the beginning for the example of sucking-off blood in operating fields.

### SUMMARY OF THE INVENTION

The present invention relates to a suction tip for suctioning a biological fluid. The suction tip comprises a main body having a main axis and a wall, a connector for a suction line, which is formed at the main body on the main axis and which defines a free connection cross section of the suction tip, and a plurality of decentral suction holes which laterally enter in the main body, which extends through the wall of the main body and which communicate with the connector within the main body. A free minimum cross section of each decentral suction hole, in a continuous continuation of its course through the wall of the main body, completely overlaps with the free connection cross section of the suction tip.

Further, the present invention relates a method of using a suction tip suction tip comprising a main body having a main axis and a wall, a connector for a suction line, which is formed at the main body on the main axis and which defines a free connection cross section of the suction tip, and a plurality of decentral suction holes which laterally enter in the main body, which extends through the wall of the main body and which communicate with the connector within the main body, wherein a free minimum cross section of each decentral suction hole, in a continuous continuation of its course through the wall of the main body, completely overlaps with the free connection cross section of the suction tip, in suctioning a body fluid of a human or animal.

Other features and advantages of the present invention will become apparent to one with skill in the art upon examination of the following drawings and the detailed description. It is intended that all such additional features and advantages be included herein within the scope of the present invention, as defined by the claims.

### BRIEF DESCRIPTION OF THE DRAWINGS

The invention can be better understood with reference to the following drawings. The components in the drawings are not necessarily to scale, emphasis instead being placed upon clearly illustrating the principles of the present invention. In the drawings, like reference numerals designate corresponding parts throughout the several views.

FIG. 1 is a longitudinal section through a first embodiment of the section tip according to the invention.

FIG. 2 is a side view of the suction tip according to FIG. 1 but with a suction tip which is rotated by 45° about its main axis as compared to FIG. 1.

FIG. 3 is a longitudinal section of the second embodiment of the suction tip according to the invention.

FIG. 4 is a longitudinal section through a further embodiment of the suction tip according to the invention; and

FIG. 5 illustrates the overlap of free minimum cross sections of decentral suction holds with a free connection cross section of the suction tip.

### DETAILED DESCRIPTION

In a suction tip for suctioning a biological fluid, the suction tip comprising a main body, having a main axis and a wall, a connector for a suction line, the connector being formed at the main body on the main axis and defining a free connection cross section, and a plurality of decentral suction holes extending through the wall of the main body and communicating in the main body with the connector, a free minimum cross section of each decentral suction hole, in a continuous continuation of its course through the wall of the main body, completely overlaps with the free connection cross section of the suction tip.

As already remarked above, the biological fluid for which the suction tip according to the present disclosure is provided, may be any fluid which is of biological origin or which includes biological material like biological cells, particularly living biological cells or larger organic molecules, like for example antibodies, oligopeptides, proteins or DNA, which are relevant in biological processes.

The plurality of the decentral suction holes through the wall of the main body of the suction tip according to the present disclosure are decentral insofar as they are entering laterally, i.e. under an angle clearly greater than zero to the main axis into the main body so that they are not directly opposing the connector for the suction line centrally formed or provided on the main axis.

A continuous continuation of the course of the respective decentral suction holes through the wall of the main body is an extrapolation of the course of the respective decentral suction hole through the wall, through any cavity in the main body up into the region of the main axis of the main body. With a straight course of the decentral suction holes through the wall, its continuous continuation is also straight. With a curved course with a constant radius of curvature, the continuous continuation keeps that radius of curvature. If the course of the respective decentral suction holes through the wall displays a varying curvature, its continuous continuation keeps the rate of change of the varying curvature.

The fact that the free minimum cross section of each decentral suction hole, in the continuous continuation of its course through the wall of the main body completely overlaps with the free connection cross section of the suction tip defined by the connector implies that the continuous continuation of the course of each decentral suction hole leads into the connector of the suction tip according to the present disclosure.

The free connection cross section of the suction tip defined by the connector is the free cross section of the connector itself or of the suction line to be connected thereto, depending on which cross section is smaller and thus defines the free connection cross section of the suction tip in operation of the suction tip. Even if this is a free cross section of the suction line, it is defined by the connector provided for the suction line.

The free minimum cross section of each decentral suction hole is its smallest free cross section over its course and perpendicular to its course through the wall of the main body. When this free minimum cross section is projected along the continuous continuation of the course of the respective decentral suction hole through the wall of the main body on the connector, it completely overlaps with the free connection cross section of the suction tip. This means that the biological fluid entering through the free minimum cross section of each decentral suction hole gets along this course to the and through the connection cross section of the suction hole without hitting on any flow obstacles. These are optimum preconditions for a laminar flow of the biological fluid through the suction tip and for minimizing the shearing forces exerted on the components of the biological fluid flowing through the suction tip meaning a protection of the integrity of the biological fluid. In this way, the danger of mixing the biological fluid flowing through the suction tip with ambient air in form of air bubbles is also minimized. Further, a reduced noise development in suctioning the biological fluid is noticed which is both advantageous as such and an indication of suctioning conditions conserving the biological fluid.

Preferably, a sum of areas of the free minimum cross sections of the decentral suction holes is at least as large as an area of the free connection cross section of the suction tip. This means that the biological fluid sucked into the suction tip is further accelerated within the suction tip, wherein the underpressure which is applied for suctioning the biological fluid successively decreases and thus not abruptly drops upon entry of the biological fluid into the suction holes. The decrease of the underpressure and the associated acceleration of the biological fluid in the main body of the suction tip are non-critical in the suction tip according to the present disclosure, because the flow of the biological fluid does not hit any obstacles within the main body of the suction tip. Further, the comparatively high sum of the areas of the free minimum cross sections of the decentral suction holes inhibit a tendency of the suction tip according to the present disclosure to adhering to tissue which is adjacent to a pool of the biological fluid.

Particularly, the decentral suction holes may enter into the main body on at least one virtual ring around the main axis. Here, a sum of the areas of the free minimum cross sections of the decentral suction holes on each of the virtual rings around the main axis may at least be as large as the area of the free connection cross section of the suction tip. The decentral suction holes may also enter into the main body on a plurality of virtual rings around the main axis.

For a particularly steady flow of the biological fluid sucked through the suction holes through the main body it is advantageous when the free minimum cross section of the decentral suction holes in the continuous continuation of their courses through the wall of the main body are uniformly distributed over the free connection cross section of the suction tip. Thus, the partial flows of the biological fluid through the individual decentral suction holes disturb one another as little as possible.

For example, the free minimum cross sections of the decentral suction holes may be circular. Further, the decentral suction holes may enter into the main body at an acute angle of not more than 60° or of not more than 45° but also of not less than typically 15° to the main axis. Here, at least several of the decentral suction holes may have a straight course through the wall of the main body, wherein their free minimum cross sections, due to the acute angle, nevertheless completely overlap with the free connection cross section of the suction tip in a continuous continuation of this straight course.

Additionally or alternatively, several of the decentral suction holes may have a curved course through the wall of the main body. Here, a radius of curvature of the curved course, related to a hole axis of the respective hole, is preferably at least as long as a maximum diameter of the main body perpendicular to its main axis. This means that the radius of curvature of the decentral suction holes with curved course is comparatively long. This is equivalent to that the biological fluid flowing through the suction holes is not strongly deviated within the suction holes. Strong deviations could cause higher shearing forces on the components of the biological fluid.

The main body, besides the decentral suction hole, may have a central suction hole s. A free minimum cross section of the additional central suction hole may be about as large as the free connection cross section of the suction tip, but it may also deviate from the free connection cross section upwards or downwards by typically 20% at maximum.

For the reason of an as steady pressure decrease as possible and a correspondingly as steady acceleration of the flow of the biological fluid through the main body as possible, each suction hole may have an area of its free cross sections steadily decreasing towards the connector. Alternatively or additionally, a free overall cross section of the main body considering all suction holes and also any cavity in the main body may steadily, i.e. without steps and preferably also without any kinks pass into the free connection cross section of the suction tip. This also implies that all suction holes have rounded or at least beveled edges.

In one embodiment of the suction tip according to the invention, an open-cell foam or sponge is arranged in a cavity within the main body, wherein the suction holes communicate with the connectors through the open-cell sponge. This open-cell sponge may serve for defoaming the biological fluid sucked into the suction tip in a generally known way. The sponge is, however, only an option in the suction tip according to the present disclosure, which may have an unfilled cavity within the main body or no cavity within the main body that could be delimited from the suction holes, i.e. which is no part of any of the suction holes.

In a preferred use of the suction tip of the present disclosure the biological fluid to be sucked-off is a body fluid of an animal or a human. The body fluid may be suctioned during a medical intervention. Even then, however, this use of the suction tip according to the present disclosure is no method of surgically or therapeutically treating the human or animal body which may be accepted from patent protection, because the body of the respective patient is neither surgically nor therapeutically treated by suctioning the body fluid. This also applies when the biological fluid to be sucked off is blood which is sucked-off out of an operating field or processed in such a way that it can be returned to a patient at which the surgery is executed, because the surgery at the patient is no subject of the use of the suction tip.

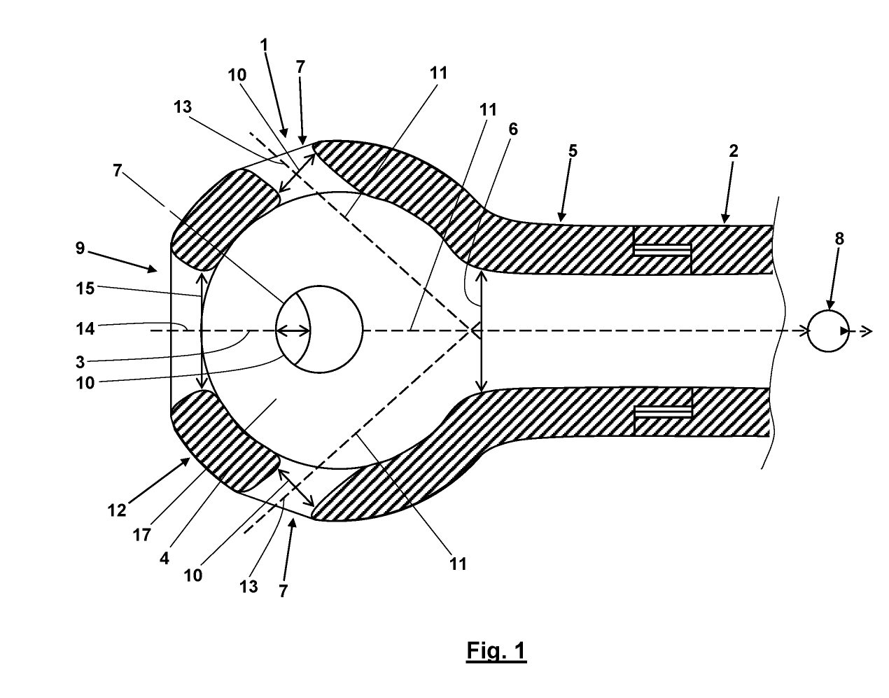

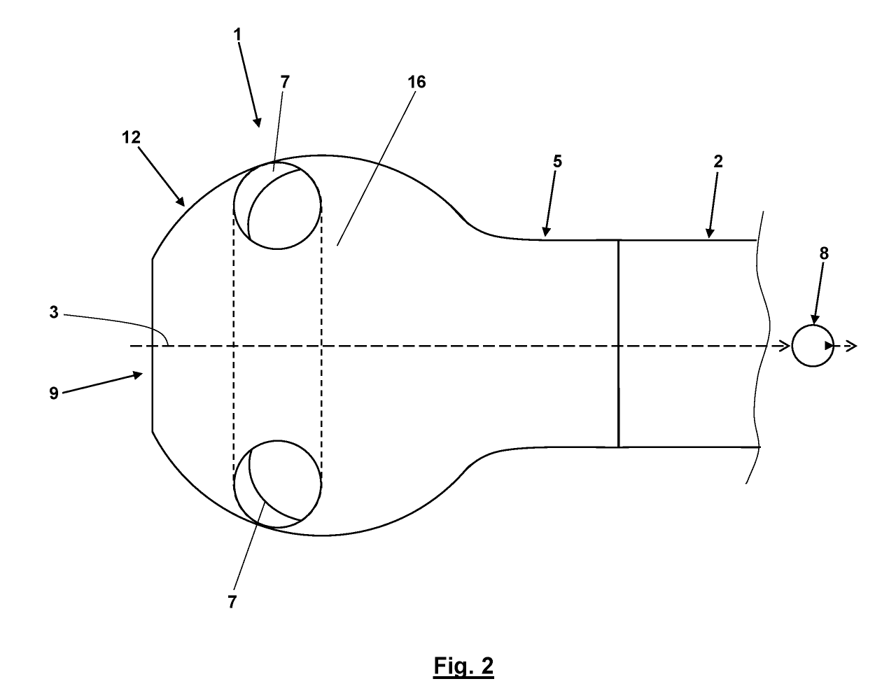

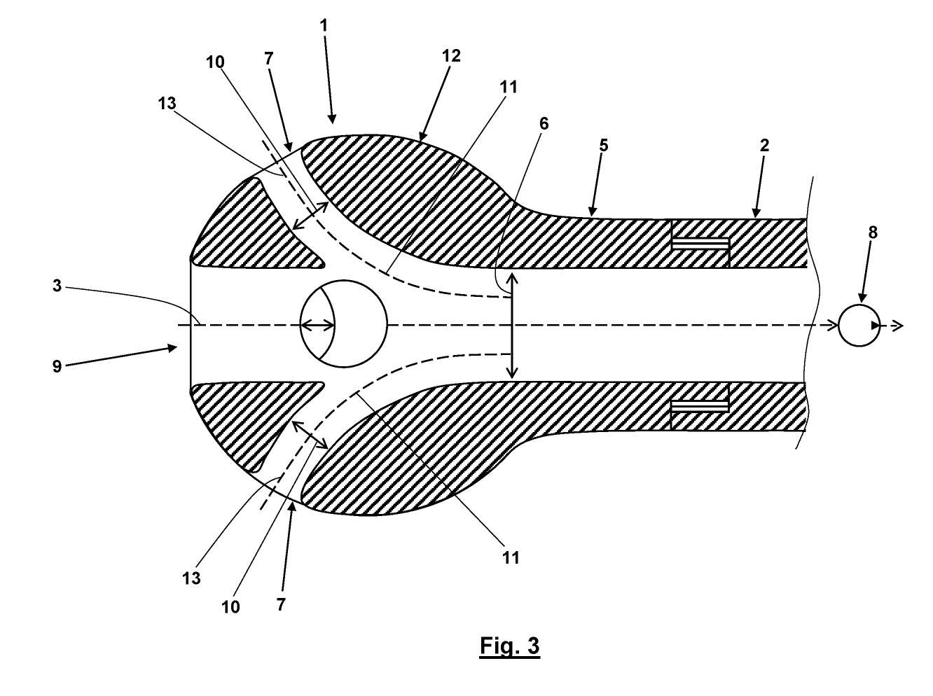

Now referring in greater detail to the drawings, the suction tip 1 depicted in FIGS. 1 and 2 serves for suctioning a biological fluid, here particularly blood, out of operating fields. The suctioning tip 1 is connected to a pump device 8 via a suction line 2. The pump device 8 via which an underpressure can be applied to the suction tip 1 is only symbolically depicted here. The underpressure may optionally be controlled depending on the signal of sensors of the suction tip 1, which are not depicted here. The suction tip 1 is specifically designed such that the sucked-off biological fluid is forwarded into the suction line 2 as gently as possible. For this purpose, particularly the following measures are taken: the suction tip 1 has a main body 12 with a main axis 3 and a wall 4. At the main body 12 a connector 5 for the suction line 2 is provided. The connector 5 defines a connection cross section 6 which is equal to the minimum cross section of the connector 5 itself and of the suction line 2 connected thereto which here pass into one another without steps and without change in cross section. A plurality of decentral suction holes 7 and a central suction hole 9 enter into the main body. The suction holes 7 and 9 extend through the wall 4 of the main body 12 and communicate with the connector 5 within the main body 12. A free minimum cross section 10 of each of the decentral suction holes 7 completely overlaps with the free connection cross section 6 of the suction tip 1, when projecting the free minimum cross section 10 along a continuous continuation 11 of the course 13 of the respective decentral suction hole 7 through the wall 4 on the connector 5. The free minimum cross section 10 is the smallest free cross section of the respective decentral suction hole 7 perpendicular to its course 13 through the wall 4. The central suction hole 9 has a straight course 14 through the wall 4 along the main axis 3, and its free minimum cross section 15 is equal to the connection cross section 6. The sum of the areas of the free minimum cross sections 10 of the decentral suction holes 7 is preferably at least as large as the area of the connection cross section 6. The course 13 of the decentral suction holes through the wall 4 of the main body 12 is straight here. Correspondingly, the continuous continuation 11 of this course is also straight. The decentral suction holes 7 are arranged on a virtual ring 16 around the main axis 3, i.e. rotational-symmetrically with regard to the main axis 3, which can particularly be seen in FIG. 2.

Whereas the main body 12 of the suction tip according to FIGS. 1 and 2 encloses a cavity 17 through which the suction holes 7 and 9 communicate with the connector 5, such a cavity in the main body 12 of the suction tip 1 that can be delimited from the suction holes 7 and 9 is not existent according to FIG. 3. Here, the free cross sections of the suction holes 7 and 9 merge in their transition into the connector 5. Further, the courses 13 of the decentral suction holes 7 through the wall 4 of the main body are not straight but curved. Correspondingly, their continuous continuations towards the connector 5 are also curved. Nevertheless it also applies here that in the projection of the free minimum cross sections 10 of the decentral suction holes 7 along the continuous continuations 11 of the courses 13 through the wall 4 result in a complete overlapping of the free minimum cross sections 10 with the connection cross section 6.

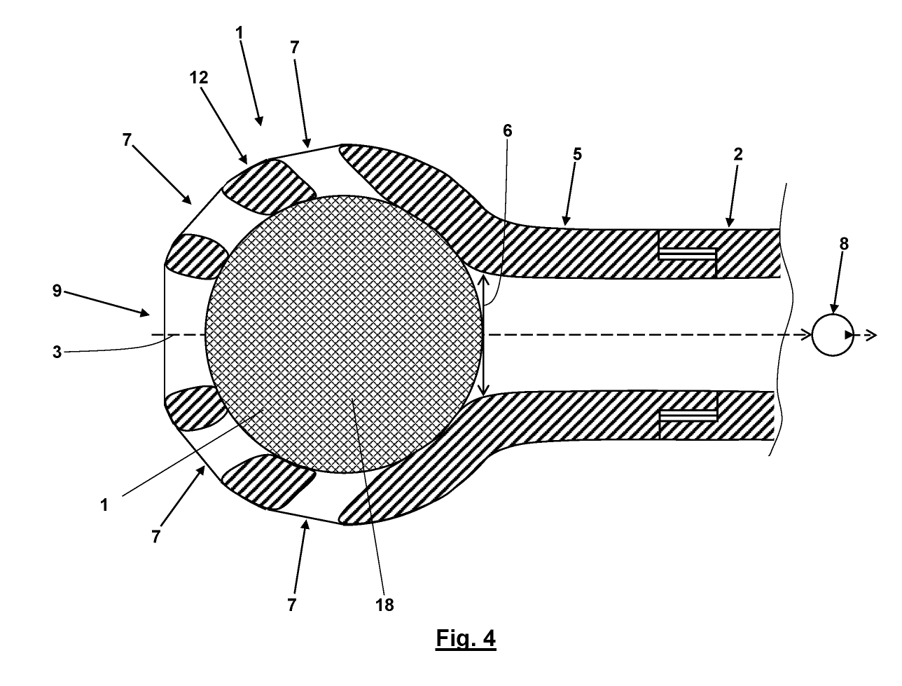

In the suction tip 1 according to FIG. 4, the main body 12 once again includes a cavity 17. Here, the cavity 17 is, however, filled with an open-cell sponge 18 through which the suction holes 7 and 9 communicate in the main body 12 with the connector 5. Further, the decentral suction holes 7 are here arranged on two virtual rings which are arranged at a distance in the direction of the main axis 3. The sum of the areas of the free minimum cross sections of the suction holes 7 of each ring is preferably at least as large as the area of the connection cross section 6.

All longitudinal sections in the FIGS. 1, 3 and 4 show that all suction holes 7 and 9, both in the regions of their entrances into the main body 12 and in the regions of their exits out of the wall 4 of the main body 12 have rounded edges.

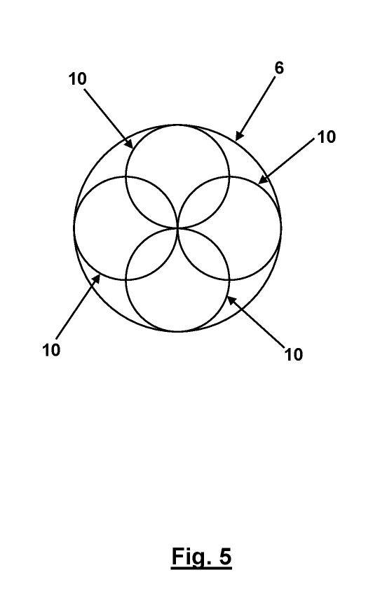

FIG. 5 illustrates an example for the overlap of the free minimum cross sections 10 of the decentral suction holes 7 of the suction tip 1 according to the present disclosure when projected onto the free connection cross section 6 along the continuous continuations 11 of the courses 13. A complete overlap of each of these free minimum cross sections 10 with the connection cross section 6 is apparent. If, as indicated here, four minimum cross sections 10 each have half the diameter of the connection cross section 6, the sum of the areas of the free minimum cross sections 10 is about as large as the area of the free connection cross section 6.

Many variations and modifications may be made to the preferred embodiments of the invention without departing substantially from the spirit and principles of the invention. All such modifications and variations are intended to be included herein within the scope of the present invention, as defined by the following claims.

We claim:

1. A suction tip for suctioning a biological fluid, the suction tip comprising
a main body having a main axis and a wall,
a connector for a suction line, which is formed at the main body on the main axis and which defines a free connection cross section of the suction tip, and
a plurality of decentral suction holes which laterally enter in the main body, which extends through the wall of the main body and which communicate with the connector within the main body,
wherein a free minimum cross section of each decentral suction hole, in a continuous continuation of its course through the wall of the main body, completely overlaps with the free connection cross section of the suction tip.

2. The suction tip of claim 1, wherein a sum of areas of the free minimum cross sections of the decentral suction holes is at least as large as an area of the free connection cross section of the suction tip.

3. The suction tip of claim 1, wherein the decentral suction holes entering in the main body are arranged on at least one virtual ring around the main axis.

4. The suction tip of claim 3, wherein a sum of areas of the free minimum cross sections of the decentral suction holes on each of the virtual rings around the main axis is at least as large as an area of the free connection cross section of the suction tip.

5. The suction tip of claim 1, wherein the free minimum cross sections of the decentral suction holes in the continuous continuations of their courses through the wall of the main body are uniformly distributed over the free connection cross section of the suction tip.

6. The suction tip of claim 1, wherein the minimum cross sections of the decentral suction holes are circular.

7. The suction tip of claim 1, wherein the decentral suction holes enter into the main body at an acute angle of not more than 60° to the main axis.

8. The suction tip of claim 7, wherein at least some of the decentral suction holes have a straight course through the wall of the main body.

9. The suction tip of claim 1, wherein at least some of the decentral suction holes have a curved course through the wall of the main body.

10. The suction tip of claim 9, wherein a radius of curvature of the curved course, related to a hole axis of the respective suction hole, is at least as long as a maximum diameter of the main body perpendicular to the main axis.

11. The suction tip of claim 1, wherein the main body further comprises a central suction hole.

12. The suction tip of claim 11, wherein a free minimum cross section of the central suction hole is equal to the free connection cross section of the suction tip.

13. The suction tip of claim 1, wherein each suction hole has an area of its free cross section which steadily decreases towards the connector.

14. The suction tip of claim 1, wherein the free total cross section of the main body steadily passes into the free connection cross section of the suction tip.

15. The suction tip of claim 1, wherein an open-cell sponge is arranged in a cavity within the main body, wherein the suction holes communicate with the connector through the open-cell sponge.

16. A method of using a suction tip suction tip comprising
a main body having a main axis and a wall,
a connector for a suction line, which is formed at the main body on the main axis and which defines a free connection cross section of the suction tip, and
a plurality of decentral suction holes which laterally enter in the main body, which extends through the wall of the main body and which communicate with the connector within the main body,
wherein a free minimum cross section of each decentral suction hole, in a continuous continuation of its course through the wall of the main body, completely overlaps with the free connection cross section of the suction tip in suctioning a body fluid of a human or animal.

17. The method of claim 16, wherein the body fluid is sucked off during a medical intervention.

18. The method of claim 16, wherein the body fluid is blood.

19. The method of claim 18, wherein the blood is sucked off in an operating field.

20. The method of claim 19, wherein the blood, after being sucked off, is processed such that it can be returned to a patient at which a surgery is executed in the operating field.
